(19) United States
(12) Patent Application Publication
(10) Pub. No.: US 2010/0256536 A1
(43) Pub. Date: Oct. 7, 2010

(54) Apparatus for treating a human or animal body by mechanical shockwaves having an exchangeable impact body

(76) Inventors: Pavel Novak, Stetten/SH (CH); Manfred Schulz, Trägerwilen/TG (CH); Klaus Froese, Konstanz (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/751,813

(22) Filed: Mar. 31, 2010

(30) Foreign Application Priority Data
Apr. 3, 2009 (DE) 20 2009 004 616.8

(51) Int. Cl. A61B 17/225

(52) U.S. Cl. 601/4

(57) ABSTRACT

The invention relates to an apparatus for a treatment of a human or animal body by mechanical shockwaves including a detection device for an automatic detection of properties of an impact body used therein.

### FIELD OF THE INVENTION

The present invention relates to an apparatus for treating a human or animal body by mechanical shockwaves.

### BACKGROUND OF THE INVENTION

Similar apparatuses are known, in particular, in the field of lithotripsy. There, body-concrements, in particular, stones in the body tissue, are disintegrated by focused mechanical shockwaves. Besides the production by electrical discharges in water, apparatuses have been developed producing the mechanical shockwaves by the collision of an accelerated projectile and an impact body and coupling said shockwaves to body tissue by means of said impact body. Such apparatuses have also been used in lithotripsy by a direct contact between the impact body or a probe connected to the impact body and the stone, and in other treatments of biological body substances. In particular, these apparatuses are used for the treatment of muscle diseases and of diseases in the transition region between muscles and bones.

An example for an apparatus of the just mentioned type is shown in EP 0 991 447. Therein, unfocused shockwaves shall be coupled into the body tissue.

In such apparatus, in many cases the intensity of the shockwave coupled-in can be varied by adjusting a pressure value of a pneumatic supply. The higher the pneumatic pressure supplied the more intense the projectile is accelerated and the higher the impulse and energy transfer to the impact body is.

Further, many apparatuses provide for an adjustability of the repeating frequency of pneumatic pulses and thus of the repeating frequency of the strokes of the projectile onto the impact body and the consequently coupled-in shockwaves.

Still further, many apparatuses provide for an exchange or replacement of an impact body with another one differing as regards geometry and/or mass.

### BRIEF SUMMARY OF THE INVENTION

The present invention has the object to provide an apparatus improved as regards the application of the impact body, an advantageous use thereof, and in particular an advantageous method by using the apparatus.

The invention relates to an apparatus for treating a human or animal body by mechanical shockwaves having a moveable projectile and an impact body, characterized by a detection device for an automatic detection of properties of said impact body.

Further, the invention relates to an advantageous use of this apparatus.

The inventors propose to design the apparatus according to the invention such that certain properties of the impact body used can be detected by the apparatus. This can relate to its type or to certain technical properties, thus to some kind of impact body class, but also to a serial number or another individual tag. Detecting an impact body class can for example relate to differentiating applicators as regards their geometrical shape, in particular the curvature of the exit surface (flat, convex or concave, and amount of curvature), or in view of their materials (hardness, sound propagation properties) or their masses.

For example, the applicator mass and the properties of an elastic suspension of the impact body, if any, together determine the oscillation properties in case of on axial displacement of the impact body after a collision of the projectile. The impact body detection can also be related to this elastic suspension, for example in that elastomer rings of different material diameter are used for which receiving recesses of different width in different impact bodies are provided.

Such a detection can result in a display of the impact body type on the apparatus so that the person responsible for the treatment has a possibility to check or has an information for setting adequate operation parameters. The apparatus can also be adapted to automatically set adequate operation parameters or at least to check the parameters set as regards the consistency with the impact body type.

The detection of an individual impact body which can thus be distinguished from another one of the same construction type as regards its individual identity, can have the function for example to guarantee a maximum number of treatments or projectile collisions. Thus, lifetime values in this regard can be predetermined and can be monitored by the apparatus in that a further operation with a certain impact body is inhibited or a warning is displayed when a maximum number stored in the apparatus has been reached.

However, the invention in its most general sense is not limited to a detection distinguishing one impact body from another one. For example, in the context of an already mentioned aspect of obeying to lifetime values, also a detection of numbers of use stored in the impact body can be performed. For example, the impact body can comprise an electronic memory that can be rewritten in a wireless manner. Thus, an apparatus according to the invention could detect and monitor the treatments already performed with this impact body during its use and could guarantee a limitation in this respect such as by incrementing the memory contents with each treatment by one and inhibiting a further operation when reaching a limit by the treatment number read from the memory or by displaying a warning. The impact body need not necessarily be exchangeable whereas this is preferred. Further, applications with exchangeable impact bodies are preferred in which the mounted impact body can be distinguished from others so that a proper detection of the impact body itself is performed as regards its technical type or its class or its identity in the sense of a serial number.

The determination of a certain period of use or a maximum treatment number for an impact body may also be combined with the operation parameters set and may consider the relevance of the parameters set for wear. For example, larger collision numbers can be allowed for a lower pressure in this manner. This can be done by the apparatus control and enable a more differentiated exploitation of the available complication-free lifetime of an impact body. Compared to mere observation by the user not supported from the apparatus side, wherein the user can only note the number of treatments, further advantages result thereby.

Single-use impact bodies may be a special case and may be preferred for example for reasons of hygiene and of lower costs per piece (for example for plastic mode pieces). Here, the apparatus could ensure that an impact body already used (at least by this apparatus or by apparatuses connected there-to in an information technological sense) must not be used once more for example because the person responsible for the treatment intends to reduce costs in an inadmissible manner by such further use.

Finally, the apparatus could also check by means of an impact body detection whether the impact body inserted is adequate for the apparatus at all and could inhibit the operation or issue a warning.

Principally, the invention can also be implemented without any particular adaption of the impact body. For example, the detection means might detect and distinguish magnetic properties of metallic impact bodies having sufficiently different shapes and/or metal materials, for example by an inductivity measurement, namely by using an electro-magnetic coil mounted in the remaining treatment apparatus. More generally spoken, electro-magnetic properties such as the electric conductivity of the impact body can be used for detection. Naturally, this also applies to other properties that the impact body anyway has such as its mass.

Preferred is a marking of the impact body, though, namely an implementation or device provided for the purpose of detection. Therein, a fastening means for the impact body such as the applicator cap of the embodiments shall be regarded to be an impact body part in the sense of marking in the following if it is replaced together with the impact body, i.e. if it is allocated to the impact body. As regards the function of detection, a detection of a fastening means allocated to the impact body and the marking of the impact body itself are equivalent, then. In this sense, the term “impact body” can mean an entity of the impact body and the fastening means together in the following wherein the marking can be located in the fastening means.

A group of markings according to the invention has at least one electrical contact, for example for measuring the electrical resistance of the impact body or of a resistor on the impact body. This resistor may be an additional conductor, for example, mounted to the outside of the impact body such as a conducting strip along a portion thereof.

In the embodiment, a ring-shaped conductor strip is shown that runs along a circumference portion of the impact body.

Another group of markings can be read by light in a general sense, i.e. including infrared light. For example, a code can be read by light which code consists of a certain sequence of more or less reflecting surfaces, in the case of visible light for example a strip consisting of bright and dark areas such as a so called bar code.

In particular in the detection of markings by light but also independent thereof, it can be necessary and desired to mount the impact body in a certain orientation as regards its longitudinal axis. For determining such an orientation, form closing elements can be used such as projecting parts engaging into recesses made on the impact body. Then, the impact body can be mounted only in a certain desired orientation (or in a plurality of respectively adequate orientations).

Another possibility is a magnet, for example in the impact body, and a corresponding magnetic sensor in the remaining apparatus (or vice versa).

For a reading by light, namely an optical reading, an optical conductor can be used, preferably between an outer casing of the apparatus an inner tube for guiding the projectile. For illustration reference is made to the embodiments.

A further possibility for detecting markings is to use electro-magnetic waves such as radio frequency waves. For this purpose, the marking may comprise a transponder and the detection means may comprise a receiving/transmitting coil adapted to determine the type of transponder. The receiving/transmitting coil can for example be arranged between the already mentioned inner tube and the already mentioned outer casing, in which respect reference is made to the embodiments, again. The receiving/transmitting coil may also be housed in a basic apparatus serving for supplying a mobile hand apparatus and being connected to the hand apparatus by a conduit. In this case, it can be necessary to bring the impact body before its mounting or the hand apparatus including the already mounted impact body near to the basic apparatus to enable a detection of the marking, i.e. a reading of the transponder.

Thus, a plurality of useable and mutually exchangeable impact bodies is part of a preferred equipment of an apparatus according to the invention although the invention can already be implemented in using only one respective impact body but detecting a new impact body in case of a replacement, i.e. in case of a definite and lasting dismounting and remounting of the treatment apparatus. Naturally, the preferred equipment of the treatment apparatus includes a plurality of impact bodies including the already-mentioned single-use impact bodies and those having a limited lifetime and are present in a stock of so to say consumable material.

Further, the impact body can be made of a plurality of parts and can in particular comprise a cap or similar device to be arranged between the impact body and the body to be treated during the treatment, the reason being hygiene, an impedance fitting, or avoiding to hurt or detriment the skin. The marking and the detection relates to a unitary set or entity of a plurality of parts as far as they are exchangeable and have to be distinguished from other units.

In particular, the impact body detection can result in a display of a detected type of impact body. Thereby, a user can check the impact body type and/or to adapt certain parameters of use, apparatus parameters or other conditions of use thereto.

Moreover, the apparatus can preferably be adapted for an automatic setting of adequate operation parameters for an impact body type detected or for a respective check of operation parameters set (and to give a signal or to inhibit the operation in case of contradictions). In particular the apparatus can be adapted to monitor that a maximum operation number of the impact body, which is regarded as an operation parameter as well, is obeyed to, and to inhibit an extension of a predetermined maximum operation number by a signal display or blocking the operation of the impact body.

Further, the application of the apparatus for the treatment of soft body tissue, for example muscles or tendons, is particularly preferred. This includes the treatment of regions near to the bones and shockwave acupuncture. Typical indications are insertion tendonitis and other applications in orthopaedics and surgery as shoulder calcifications, heel pain, pseudarthroses, but also muscle pain. Further indications are in neurology such as the improvement of the mobility after strokes, the treatment of post-traumatic spasm and polyneuropathies. Within urology, for example the chronic pelvic pain syndrome can be treated; in angiology/dermatology and surgery also scars or skin burns can be treated as well as improvements of wound healing can be reached.

The invention will be explained in reference to some exemplary embodiments wherein the individual features may be relevant for all claim categories named and also in other combinations than shown.

### BRIEF DESCRIPTION OF THE DRAWINGS

FIG. 1 shows an apparatus according to the invention in longitudinal section wherein details of the invention are not shown in FIG. 1.

FIG. 2-9 respectively show a part of FIG. 1 including additionally illustrated detecting means and/or markings, and thus a first to eighth embodiment.

### DETAILED DESCRIPTION OF THE INVENTION

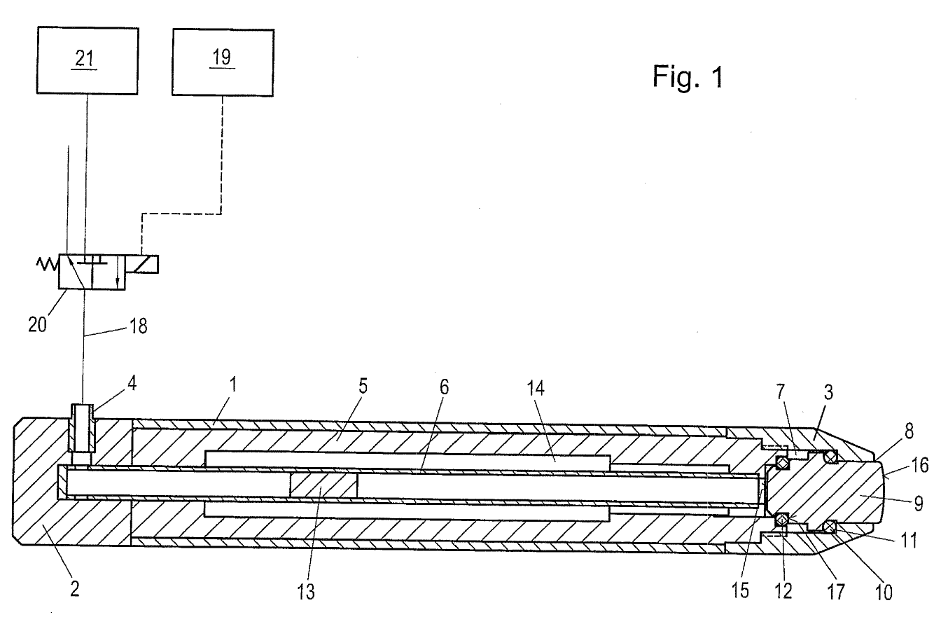

FIG. 1 shows an apparatus for coupling focused mechanical shock waves into for example the human body is shown as a section along a longitudinal axis. A tube portion constitutes a casing 1 being closed at the respective ends by an air supply cap 2 distal from the body during application and an applicator cap 3 proximal to the body during application.

Air supply cap 2 comprises a pressurized air terminal 4 for a pneumatic supply. In a manner known as such, a valve 20 controlled by a control unit 19 via a pneumatic supply line 18 is connected to pressurized air terminal 4, in particular a magnetic valve, that couples in pressurized air pulses via the pressurized air terminal in a steady iteration rate between approximately 1 Hz and 50 Hz.

The apparatus is implemented as a device to be held manually by an operating person, which device is connected to a base station including control unit 19 and a compressor 21 by pneumatic line 18 mentioned and which can be positioned on the patient manually. It serves for the treatment of soft tissue, in particular muscles.

The details of the pneumatic supply are not relevant for this invention and are familiar to an expert as part of the prior art. Preferably, the frequency is adjustable. The iterative operation can be more complex than a simple steady repetition of pulses with a certain frequency, in particular also with a multiplicity of succeeding collisions in relatively short time distances, that is with a relatively high frequency, wherein groups of such collisions in these short time distances are mutually separated by somewhat longer time distances. Details of this aspect are not relevant for this invention but can be combined therewith.

A guiding tube 6 is supported within casing 1 by an insert 5 whose end distal from the body during application ends in air supply cap 2 and communicates with pressurized air terminal 4 there. The end of the guiding tube 6 proximal to the body during the application ends in a part of insert 5 projecting into applicator cap 3, namely short before the end of insert 5 there and an inner space 7 in applicator cap 3.

An impact body 9 is received in inner space 7 communicating with an applicator opening 8 being distal from the body during application, by two radial shoulders. Impact body 9 is supported on one of the radial shoulders by an O-ring 10 of an elastomer and has a flange 11 hereto. An end 15 of impact body 9 facing away from the body is supported on insert 5 by a further O-ring 12, namely on a front face encircling the already mentioned end of insert 5. Therein, O-ring 12 is positioned between this front face and a flange 17 or a shoulder of impact body 9. Applicator opening 8 serves for guiding impact body 9 in a manner displaceable in the longitudinal direction and fixes impact body 9 transverse to the longitudinal direction. The axial displaceability is limited by the resilience of elastomer rings 10 and 12 and is substantially higher than 0.6 mm relative to the rest of the apparatus in case of an operation of the apparatus in air.

The features of impact body 9 being the applicator to be positioned onto the skin will be discussed in further detail below. It can be replaced by unscrewing the applicator cap.

A projectile 13 is inserted into the adjacent region of guiding tube 6 and is in contact with impact body 9 in FIG. 1. it fits radially with a small clearance (as regards the guiding tube and the substantially cylindrical geometry of projectile 13). Projectile 13 can be moved in guiding tube 6 by pressure differences of the air column in guiding tube 6 before and behind it (i.e. right and left of projectile 13 in FIG. 1), and can in particular be accelerated onto impact body 9. Hereto, it is accelerated from a starting position (not shown) in the left side in FIG. 1 by a pressurized air pulse via pressurized air terminal 4 and collides with impact body 9 by its front surface (not numerated in FIG. 1 for clarity of the drawing) facing impact body 9.

The back movement of projectile 13 is performed by a back flow of air from a pressure chamber 14 around guiding tube 6 within insert 5. During the acceleration of projectile 13 towards impact body 9, the air is pushed therein and compressed thereby. As soon as magnetic valve 20 in pneumatic line 18 of pressurized air terminal 4 switches off the pressure, projectile 13 is moved back into the starting position. Naturally, this can also be performed by an additional or alternative pressure application of pressure chamber 14 or another air volume on the body side of projectile 13. The end of guiding tube 6 distal from the body during application ends in a magnetic holder 17 for projectile 13.

Impact body 9 has a rotationally asymmetrical cylinder shape and is defined In the axial direction by the entry surface of end 15 and the somewhat convex exit surface 16. The outer circumferential surface has already described flange-like structures 11 and 17 providing support shoulders for O-rings 10 and 12. Further, an exit side part of the cylinder shape has a constant radius and is thus axially displaceable within opening 8.

Impact bodies such as impact body 9 shown here can be substantially different as regards shape, material, and suspension. For example there are different focusing and non-focusing shapes, compare for example the rotational ellipsoid shape in DE 10 2007 013 288 and various curved exit surfaces in the prior art. Further, various materials are considered such as stainless steel, titanium, and different ceramics such as silicon nitride, as well as plastics. Finally, different impact bodies can be suspended with varying hardness and can perform differing displacements, i.e. macroscopic movements of different length in the process of coupling-in the shockwave.

Various embodiments different in this respect are particularly well adapted for certain applications, respectively, such as for acupuncture, enthesis treatments, muscle treatments, trigger point treatments etc. Herein, various parameters are used for example for the driving pressure or the pulse frequency, which depend on the impact body used and the indication. Thus, there is a substantial advantage with respect to a reasonable system control if an automatic detection of the impact body type is performed.

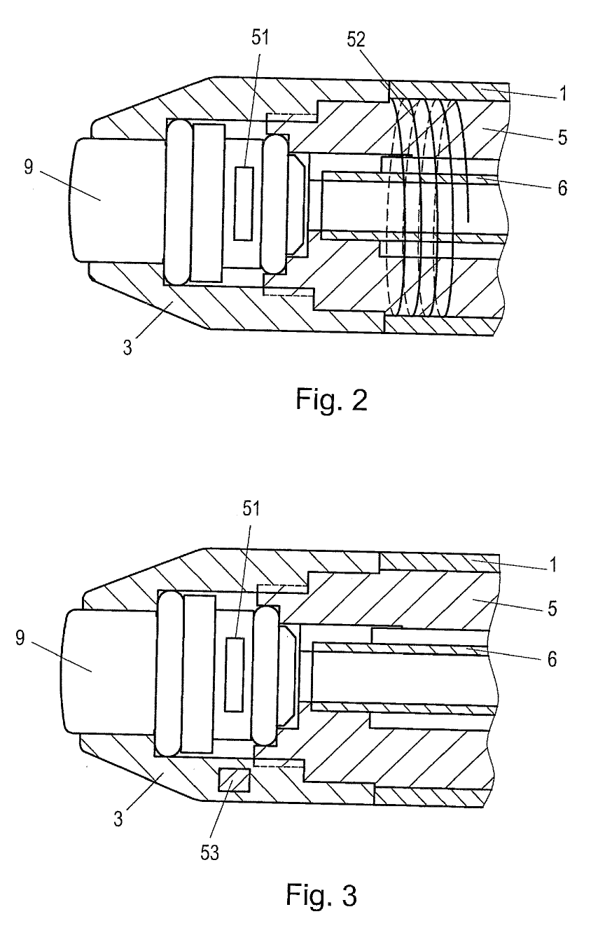

FIG. 2 shows a first embodiment having an impact body detection as a detail of FIG. 1 (inverse) including additionally illustrated details of the invention. FIG. 2 shows a so called RFID (radio-frequency identification) transponder 51 being mounted on the impact body 9. 52 is a receiving/transmitting coil illustrated only symbolically in the hand part of FIG. 1. It is mounted between the outer casing 1 and the insert 5 and as near as possible to the impact body 9 and the RFID transponder 51 mounted thereto, i.e as far as possible to the left side in FIG. 2. The figure shows that when dismounting the applicator cap 3, the region in which the receiving/transmitting coil 52 is mounted is not opened, though, so that the coil is not endangered in this respect. Due to this proximity, the detection of the RFID transponder 51 is alleviated. In particular, a coupling to other impact bodies which can lie on a table in a treatment room near to the hand part, for example, is as weak as possible relative to the coupling to the RFID transponder 51 shown, thus.

RFID detection systems are known as such. The RFID transponder 51 is energy supplied and read via an electromagnetic high frequency field of typically 13.56 MHz. Since only short ranges are necessary here, the system requires low power, only. Further, the RFID-transponder 51 can be very small and does not affect the performance of the impact body 9.

FIG. 3 shows a second embodiment as regards the detection in an analogues manner. Again, RFID transponder 51 is provided, whereas the receiving/transmitting coil is housed in the basic apparatus not shown, compare FIG. 1. In such embodiments of the invention, the user generally must approach the impact body 9 to be inserted and not yet mounted or already mounted, i.e. the body-side end of the hand part, to the basic apparatus to enable a detection. In this embodiment, however a further improvement is included in that the risk that accidentally an impact body in the proximity of the basic apparatus but not to be used, is detected, can be excluded. Hereto, this embodiment provides a permanent magnet 53 in the applicator cap 3 holding the impact body 9 to the hand part. The RFID transponder 51 can detect the presence of the permanent magnet 53 and can thus distinguish the mounted condition of the impact body 9 from a non-mounted condition. The RFID transponder is so to say enabled by the detection of the permanent magnet 53.

In this context, it can be advantageous not to mount the RFID transponder 51 independently of the permanent magnet to the impact body, as shown, but to a border region establishing a spacial proximity as close as possible between the permanent magnet 53 and the RFID transponder 51. Hereto, a non-shown form closure can be used, such as a recess in the impact body 9 and a fitting nose in the applicator cap 3 in order to establish a rotationally correct position during mounting of the impact body 9 and to inhibit an accidental arrangement of the RFID transponder 51 in another angular position than the one close to the permanent magnet 53.

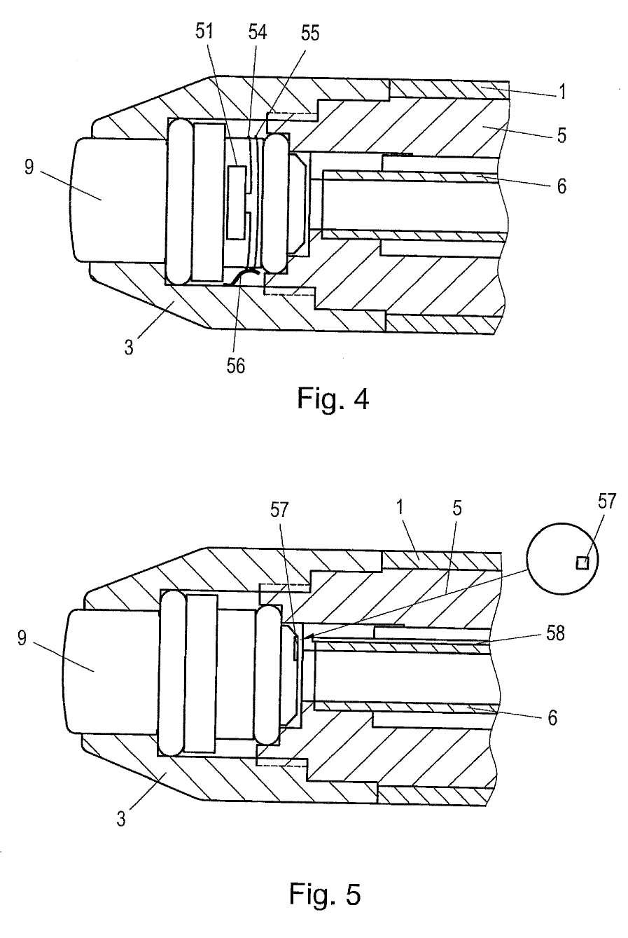

FIG. 4 shows the RFID transponder 51 of FIGS. 2 and 3 again. Here, it is connected to two ring electrodes 54 and 55 via conduits, however, which electrodes can be contacted by a resilient contact 56 in the applicator cap 3 shown in the lower portion of FIG. 4. The resilient contact 56 can short-circuit both ring electrodes 54 and 55 and can distinguish the RFID transponder 51 of the impact body 9 mounted from others in a similar manner as in FIG. 3, thus. Besides that, the explanations to FIG. 3 apply.

The next embodiment in FIG. 5 uses an optically detectable marking of the impact body 9, namely a two-dimensional barcode 54. It is eccentrically mounted to a front surface of the impact body 9 distal to the body, as shown in the small detail illustration on the right side of FIG. 5, and detectable via a glass fibre bundle 58 forming so to say a read head on its side to the marking. Hereto, the glass fibre bundle 58 can be illuminated by a light source not illustrated such as a LED or laser diode. In particular, the bar code can also be read quasi serially by scanning and using the multiplicity of glass fibres (i.e. using the glass fibres sequentially) instead of reading in one step, quasi parallel as an image, and can be analysed electronically.

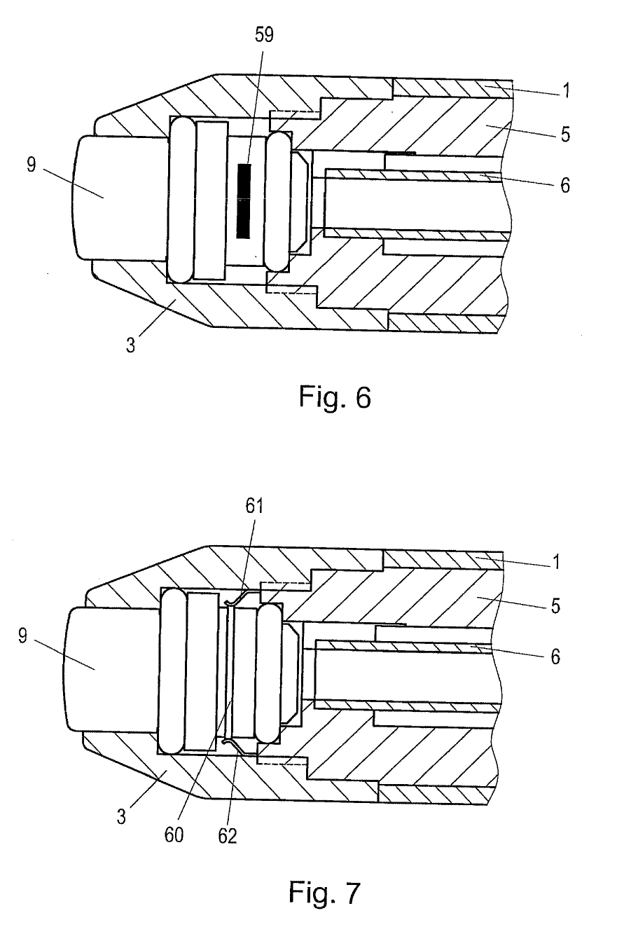

FIG. 6 shows a further embodiment in which the impact body 9 comprises a barcode 59 fixed on its circumferential surface. This barcode is illustrated in FIG. 6 only symbolically as a black strip 59. It is adapted to be detected before mounting of the impact body 9 by a reading device housed in the above mentioned basic apparatus. Here, the user shall verify that he actually mounts the impact body 9 detected and not another one, and, as a precondition, that a new detection is performed when exchanging the impact body.

The embodiment in FIG. 7 is directed to an electrical detection instead of an optical one. Hereto, there is a resistance strip 60 running around the same circumferential surface of the impact body 9 as in the previous embodiment, that can be contacted by resilient contacts 61 and 62. Different impact body types can be detected and distinguished by means of different electrical resistances of the resistance strip 60 between the contact points of the resilient contacts 61 and 62.

In an embodiment not illustrated, even memories such as an EEPROM can be used in this manner, which can be read via respective contacts.

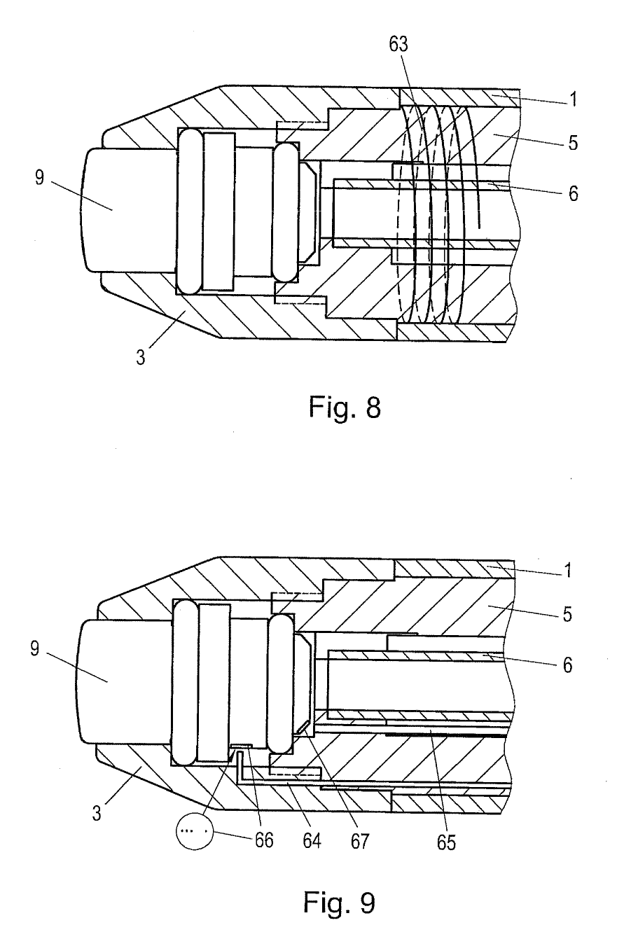

FIG. 8 again shows a coil, however, here a detector coil 63. It serves for an inductive determination of the impact body type by detecting its magnetic impedance. This is based on sufficiently clear differences between the impact body types in question as regards material and/or size and/or shape, as a precondition. As regards mounting the detector coil 63, the above explanations relating to the receiving/transmitting coil 53 in FIG. 2 apply.

The last embodiment in FIG. 9 again relates to an optical detection as in FIG. 5.

Here, two optical conductors 64 and 65 are provided similar to FIG. 5. Both are optically conducting glass fibre bundles. The last portion of the optical conductor 64 extends through the applicator cap 3 and thus comprises an optical coupling between the applicator cap 3 and the insert 5 not shown in detail.

The second optical conductor 65 is arranged more inwardly and in a similar manner as the optical conductor 58 of FIG. 5. Here, dot sequences are sampled as markings as shown by marking 66 in an exemplary manner and are provided as a marking 67 allocated to the other optical conductor 65 in a corresponding manner at the chamfer of the impact body 9. Therein, both markings and both optical conductors are alternatives which can also be provided in combination, however, and illustrate the different geometrical possibilities, in particular the arrangement on a circumferential surface as in marking 66 and the arrangement on a chamfered surface as in marking 67 (and the arrangement on a front surface as 57 in FIG. 5, naturally). For example, the four black dots illustrated together with the interspace can represent a start bit and four further information bits. The start bit can be disposed of if the mounting of the impact body 9 works sufficiently precisely as regards the rotational position. The already mentioned form closures can serve for this purpose, also in the form of inclinations on the impact body 9 enabling only one certain rotationally correct mounting because of correspondence to projections on the applicator cap 3 or the insert 5.

What is claimed is:

1. An apparatus for treating a human or animal body by mechanical shockwaves having
a moveable projectile (13) and
an impact body (9),
characterized by a detection device (51-67) for an automatic detection of properties of said impact body (9).

2. The apparatus of claim 1 wherein said impact body (9) is exchangeable and can be distinguished from other impact bodies (9) being mountable by said detection.

3. The apparatus of claim 1 wherein said impact body (9) has a marking (51, 54, 55, 57, 59, 60, 66, 67) detectable by said detection device (51-62, 64-67).

4. The apparatus of claim 3 wherein said marking (60) is detectable via an electrical contact (61, 62).

5. The apparatus of claim 4 wherein said marking is an electrical resistor (60).

6. The apparatus of claim 5 wherein said resistor (60) is a ring-shaped conductor strip around a circumference of a portion of said impact body (9).

7. The apparatus of claim 3 wherein said marking (57, 66, 67) is detectable by light including infrared light.

8. The apparatus of claim 7 wherein said marking (57, 66, 67) is a bright/dark code, in particular a barcode.

9. The apparatus of claim 7 wherein said impact body (9) and a part of the remaining apparatus have complementary form closing elements for defining an orientation in mounting said impact body (9).

10. The apparatus of claim 7 wherein said impact body (9) and a part of the remaining apparatus have a magnet (53) and a complementary magnetic sensor for defining an orientation in mounting said impact body (9).

11. The apparatus of claim 7 having an optical conductor (58, 64, 65) for detecting said marking, mounted in said apparatus, namely between an inner tube (6) for guiding said projectile (13) and an outer casing (1) of said apparatus.

12. The apparatus of claim 3 wherein said marking (51) is readable by radio frequency waves.

13. The apparatus of claim 12 wherein said marking comprises a transponder (51) and said detection device comprises a receiving/transmitting coil (52).

14. The apparatus of claim 13 wherein said receiving/transmitting coil (52) is arranged between an inner tube (6) for guiding said projectile (13) and an outer casing (1) of said apparatus.

15. The apparatus of claim 12 having a receiving/transmitting coil (52) of said detection device arranged in a basic apparatus supplying a hand apparatus of said apparatus.

16. The apparatus of claim 1 wherein said impact body (9) has no marking and said detection device (63) detects said impact body by electro-magnetic properties, in particular by a magnetic inductivity measurement.

17. The apparatus of claim 1 comprising a plurality of mutually exchangeable impact bodies (9).

18. The apparatus of claim 1 being adapted to display the type of said impact body (9) upon its detection.

19. The apparatus of claim 1 being adapted to check or automatically set operation parameters adapted to a type of said detected impact body (9) for said treatment.

20. The apparatus of claim 1 being adapted to monitor a maximum operation number of said impact body (9) by means of said detection of said impact body (9).

21. A method of using the apparatus of claim 1 comprising operating said apparatus for treating the human or animal body.
